(19) United States
(12) Patent Application Publication
(10) Pub. No.: US 2025/0009778 A1
(43) Pub. Date: Jan. 9, 2025

(54) KRAS ANTAGONISTS

(71) Applicant: Truetiva, Inc., Grand Rapids, MI (US)

(72) Inventor: J. Phillip Kennedy, Grand Rapids, MI (US)

(21) Appl. No.: 18/892,022

(22) Filed: Sep. 20, 2024

Related U.S. Application Data
(63) Continuation of application No. 17/449,275, filed on Sep. 29, 2021.
(60) Provisional application No. 63/198,448, filed on Oct. 20, 2020.

(51) Int. Cl. A61K 31/7068; A61K 31/282; A61K 31/337; A61K 31/4745; A61K 31/513; A61K 33/243; A61K 45/06; A61P 35/00

(52) U.S. Cl. CPC A61K 31/7068; A61K 31/282; A61K 31/337; A61K 31/4745; A61K 31/513; A61K 33/243; A61K 45/06; A61P 35/00

(57) ABSTRACT

Compounds capable of binding to and inhibiting the activity of RAS protein are described herein along with compositions and methods useful for treating RAS protein related diseases such as, for example, cancer.

### CROSS-REFERENCE TO RELATED APPLICATIONS

This application claims priority from U.S. Provisional No. 63/198,448, entitled “KRAS Antagonists,” filed Oct. 20, 2020, the entirety of which is hereby incorporated by reference.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Mutations in RAS proteins are associated with about 16% of all human cancers, and mutations in KRAS account for nearly 85% of all RAS-related cancers. Mutant KRAS is therefore a highly sought-after anticancer drug target. Nonetheless, despite decades of effort RAS proteins in general and KRAS in particular are considered undruggable.

Cellular KRAS is tethered to the inner surface of the plasma membrane by a farnesylated polybasic lipid anchor and cycles between active guanosine triphosphate (GTP)- and inactive guanosine diphosphate (GDP)-bound conformational states. KRAS is activated by epidermal growth factor receptors that recruit guanine nucleotide exchange factors (GEFs) are recruited to KRAS and initiate exchange of GDP for GTP while GTPase activating proteins (GAPs) facilitate hydrolysis of GTP by KRAS activated. Active KRAS interacts with effectors such as Raf in the MAPK pathway and PI3K in the AKT pathway, driving cell growth and proliferation. In a regulated RAS cycle, signaling is turned off upon GTP hydrolysis. Oncogenic mutations that impair its GAP-mediated or intrinsic GTPase activity render KRAS constitutively active, thereby causing uncontrolled cell growth/proliferation, leading to cancer.

Conservation of the nucleotide-binding site among a diverse group of small GTPases and the high (picomolar) affinity of RAS for its endogenous ligands, GDP or GTP, have made competitive inhibition that avoids off-target effects difficult. Proof-of-principle studies have resulted in several allosteric small-molecule KRAS binders, and a number of recent reports describing molecular fragments, small molecules, peptidomimetics, and monobodies that bind KRAS and modulate its functions in various ways. Non-covalent allosteric inhibition of KRAS is necessary to target mutations in KRAS including G12D, G12V, G13D, and Q61H found in biliary tract, small intestine, colorectal, lung, and pancreatic cancers, which account for greater than 78% of all KRAS-associated cancers.

### SUMMARY OF THE INVENTION

Various embodiments are directed to compositions for treating cancers associated with RAS and KRAS mutations, comprising a therapeutically effective amount of a compound of general Formula I or stereoisomers, salts, and solvates thereof:

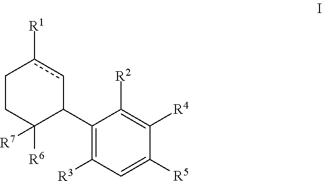

wherein:
R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres;
R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres;
R⁴ is hydrogen, hydroxyl, halogen, substituted or unsubstituted alkyl, carboxyl, alkoxy, aryl, aryloxy, arylalkyl, or pharmaceutically acceptable isosteres;
R⁵ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; and
R⁶ and R⁷ are each, individually, hydrogen, hydroxyl, halogen, substituted or unsubstituted alkyl, substituted or unsubstituted alkenyl, substituted or unsubstituted alkynyl, carboxyl, alkoxy, aryl, aryloxy, arylalkyl, or amino, or pharmaceutically acceptable isosteres;
wherein cycloalkyl moieties can include optional double bonds indicated by dashed lines;
a therapeutically effective amount of a chemotherapeutic agent; and
a pharmaceutically acceptable excipient.

In some embodiments, the chemotherapeutic agent may be metformin, phenformin, buformin, imatinib, nilotinib, gefitinib, sunitinib, carfilzomib, salinosporamide A, retinoic acid, cisplatin, carboplatin, oxaliplatin, mechlorethamine, cyclophosphamide, chlorambucil, ifosfamide, azathioprine, mercaptopurine, doxifluridine, fluorouracil, gemcitabine, methotrexate, tioguanine, vincristine, vinblastine, vinorelbine, vindesine, podophyllotoxin, etoposide, teniposide, tafluposide, paclitaxel, docetaxel, irinotecan, topotecan, amsacrine, actinomycin, doxorubicin, daunorubicin, valrubicin, idarubicin, epirubicin, plicamycin, mitomycin, mitoxantrone, melphalan, busulfan, capecitabine, pemetrexed, epothilones, 13-cis-retinoic acid, 2-CdA, 2-chlorodeoxyadenosine, 5-azacitidine, 5-fluorouracil, 5-FU, 6-mercaptopurine, 6-MP, 6-TG, 6-thioguanine, abraxane, accutane®, actinomycin-D, adriamycin®, adrucil®, afinitor®, agrylin®, ala-cort®, aldesleukin, alemtuzumab, ALIMTA, alitretinoin, alkaban-AQ®, alkeran®, all-transretinoic acid, alpha interferon, altretamine, amethopterin, amifostine, aminoglutethimide, anagrelide, anandron®, anastrozole, arabinosylcytosine, ara-C, aranesp®, aredia®, arimidex®, aromasin®, arranon®, arsenic trioxide, arzerra™, asparaginase, ATRA, avastin®, azacitidine, BCG, BCNU, bendamustine, bevacizumab, bexarotene, BEXXAR®, bicalutamide, BiCNU, blenoxane®, bleomycin, bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Citrovorum Factor, Cladribine, Cortisone, Cosmegen®, CPT-11, Cytadren®, Cytosar-U®, Cytoxan®, Dacarbazine, Dacogen, Dactinomycin, Darbepoetin Alfa, Dasatinib, Daunomycin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab, ozogamicin, Gemzar Gleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICER, Toposar®, Topotecan, Toremifene, Torisel®, Tositumomab, Trastuzumab, Treanda®, Tretinoin, Trexall™, Trisenox®, TSPA, TYKERBR, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xeloda®, Zanosar®, Zevalin™, Zinecard®, Zoladex®, Zoledronic acid, Zolinza, Zometa®, and combinations thereof.

In some embodiments, the chemotherapeutic agent may be gemcitabine, paclitaxel, cisplatin, oxaliplatin, 5-fluorouracil, capecitabine, and irinotecan. In some embodiments, the therapeutically effective amount of the compositions may be about 0.01 to about 100 mg/kg body weight. In some embodiments, the composition may contain about 40 mg to about 800 mg of a compound of Formula 1, and in certain embodiments, the composition may contain about 40 mg to about 800 mg of a chemotherapeutic agent.

Other embodiments are directed to methods for treating cancer including the step of administering to a patient in need of treatment a therapeutically effective amount of a compound of general Formula I or stereoisomers, salts, and solvates thereof. In some embodiments, such compositions may further include a therapeutically effective amount of a chemotherapeutic agent. In particular embodiments, the chemotherapeutic agent may be gemcitabine, paclitaxel, cisplatin, oxaliplatin, 5-fluorouracil, capecitabine, or irinotecan. In some embodiments, a therapeutically effective amount of compound of general Formula I or stereoisomers, salts, and solvates thereof and a chemotherapeutic agent may be about 0.01 mg/kg to about 100 mg/kg of subject body weight per day. In some embodiments, the method may further include repeating the step of administering the compound.

In various embodiments, the cancer being treated may be melanoma, liposarcoma, lung cancer, breast cancer, prostate cancer, pancreatic cancer, leukemia, kidney cancer, esophageal cancer, brain cancer, lymphoma, colon cancer, and combinations thereof, and in certain embodiments, the cancer being treated may be pancreatic cancer. In some embodiments, the cancer may be a KRAS associated disease such as, for example, hepatocellular carcinoma, LKB1 mutant cancers, LKB1 loss of heterozygosity (LOH) driven cancers, KRAS mutant cancers, Peutz-Jeghers syndrome (PJS), Cowden's disease (CD), tuberous sclerosis (TS), and combinations thereof.

Further embodiments are directed to compositions containing a therapeutically effective amount of a chemotherapeutic agent selected from the group consisting of gemcitabine, paclitaxel, cisplatin, oxaliplatin, 5-fluorouracil, capecitabine, and irinotecan; and a therapeutically effective amount of a compound of general Formula I or stereoisomers, salts, and solvates thereof. In some embodiments, the therapeutically effective amount of the composition may be about 0.01 to about 100 mg/kg body weight. In certain embodiments, the composition may include about 40 mg to about 800 mg of a compound of Formula 1, and in some embodiments, the composition may include about 40 mg to about 800 mg of a chemotherapeutic agent.

### DESCRIPTION OF THE DRAWINGS

Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all of these specific details. In other instances, well known process operations have not been described in detail so as to not unnecessarily obscure the present invention.

FIG. 1A is a bar graph showing fluorescence resulting from KRAS (G12C) nucleotide exchange assay for example compound TV-1001.

FIG. 1B is a bar graph showing fluorescence resulting from KRAS (G12C) nucleotide exchange assay for example compound TV-1002.

FIG. 1C is a bar graph showing fluorescence resulting from KRAS (G12C) nucleotide exchange assay for example compound TV-1003.

FIG. 1D is a bar graph showing fluorescence resulting from KRAS (G12C) nucleotide exchange assay for example compound TV-1004.

FIG. 2 is a table showing IC50 data for each of example compounds TV-1001, TV-1002, TV-1003, and TV-1004 based on in vitro cell viability of pancreatic cancer cell lines, Capan-2, Panc 10.05, CFPAC-1, HPAF-II, SW 1990, BxPC-3, and AsPC-1.

FIG. 3A is a table showing the increase or decrease is cell killing resulting from administration of a combination of each of example compounds TV-1001, TV-1002, TV-1003, and TV-1004 and chemotherapeutics; gemcitabine, paclitaxel, cisplatin, oxaliplatin, 5-fluorouracil, capecitabine, and irinotecan, to Capan-2 cells.

FIG. 3B is a table showing the increase or decrease is cell killing resulting from administration of a combination of each of example compounds TV-1001, TV-1002, TV-1003, and TV-1004 and chemotherapeutics; gemcitabine, paclitaxel, cisplatin, oxaliplatin, 5-fluorouracil, capecitabine, and irinotecan, to Panc 10.05 cells.

FIG. 3C is a table showing the increase or decrease is cell killing resulting from administration of a combination of each of example compounds TV-1001, TV-1002, TV-1003, and TV-1004 and chemotherapeutics; gemcitabine, paclitaxel, cisplatin, oxaliplatin, 5-fluorouracil, capecitabine, and irinotecan, to CFPAC-1 cells.

FIG. 3D is a table showing the increase or decrease is cell killing resulting from administration of a combination of each of example compounds TV-1001, TV-1002, TV-1003, and TV-1004 and chemotherapeutics; gemcitabine, paclitaxel, cisplatin, oxaliplatin, 5-fluorouracil, capecitabine, and irinotecan, to HPAF-II cells.

FIG. 3E is a table showing the increase or decrease is cell killing resulting from administration of a combination of each of example compounds TV-1001, TV-1002, TV-1003, and TV-1004 and chemotherapeutics; gemcitabine, paclitaxel, cisplatin, oxaliplatin, 5-fluorouracil, capecitabine, and irinotecan, to SW 1990 cells.

FIG. 3F is a table showing the increase or decrease is cell killing resulting from administration of a combination of each of example compounds TV-1001, TV-1002, TV-1003, and TV-1004 and chemotherapeutics; gemcitabine, paclitaxel, cisplatin, oxaliplatin, 5-fluorouracil, capecitabine, and irinotecan, to BxPC-3 cells.

FIG. 3G is a table showing the increase or decrease is cell killing resulting from administration of a combination of each of example compounds TV-1001, TV-1002, TV-1003, and TV-1004 and chemotherapeutics; gemcitabine, paclitaxel, cisplatin, oxaliplatin, 5-fluorouracil, capecitabine, and irinotecan, to AsPC-1 cells.

FIG. 4A is a representative synergy data showing the interaction of TV-1003 and Paclitaxel.

FIG. 4B is a representative synergy data showing ZIP synergy score data describing the interaction of TV-1003 and Paclitaxel.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering,” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments of the invention are directed to compounds that bind to and inhibit KRAS activity, and pharmaceutical compositions containing such compounds. The compounds and pharmaceutical compositions may be effective for treating cancers associated with RAS and KRAS mutations, by blocking RAS activity, slowing cell growth, proliferation, and metastasis of cancers that may otherwise be untreatable. Thus, other embodiments are directed to methods for treating cancer by administering the compounds and compositions of the invention to subjects in need of treatment.

The compounds of various embodiments include compounds of Formula I:

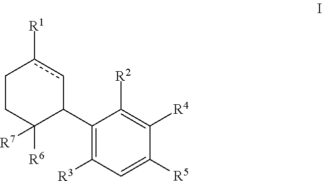

where R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres; R⁴ is hydrogen, hydroxyl, halogen, substituted or unsubstituted alkyl, carboxyl, alkoxy, aryl, aryloxy, arylalkyl, or pharmaceutically acceptable isosteres; R⁵ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; and R⁶ and R⁷ are each, individually, hydrogen, hydroxyl, halogen, substituted or unsubstituted alkyl, substituted or unsubstituted alkenyl, substituted or unsubstituted alkynyl, carboxyl, alkoxy, aryl, aryloxy, arylalkyl, amino or pharmaceutically acceptable isosteres. The cycloalkyl moiety may or may not include double bonds such as the optional double bond indicated by the dashed line. The compounds of Formula I encompass any stereoisomers, salts, and solvates thereof.

In particular embodiments, R¹, R², R³, R⁴, R⁵, R⁶ and R⁷ may each be a functional group, pro-functional group, or isostere thereof, or may be metabolised to an active functional group. In some embodiments, at least R¹, R², R⁴, and R⁵ may be hydrophobic. For example, each of R¹, R², R⁴, and R⁵ may each, individually, be hydrogen, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. In some embodiments, R¹ and R³ may be a functional group capable of making a hydrogen bond with a functional group associated with the binding pocket of a RAS protein. For example, R¹ and R³ may each, individually, be hydrogen, hydroxyl, carboxyl, amino, alkyloxy, aryloxy, ketonyl, ester, or ether.

The compounds of various embodiments include compounds of Formula II:

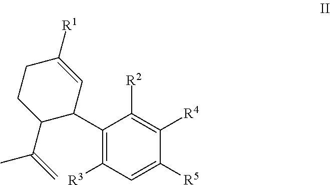

where R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres; R⁴ is hydrogen, hydroxyl, halogen, substituted or unsubstituted alkyl, carboxyl, alkoxy, aryl, aryloxy, arylalkyl, or pharmaceutically acceptable isosteres; and R⁵ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres. The cycloalkyl moiety may or may not include double bonds such as the optional double bond indicated by the dashed line. The compounds of Formula II encompass any stereoisomers, salts, and solvates thereof.

In particular embodiments, R¹, R², R³, R⁴, R⁵, R⁶ and R⁷ may each be a functional group, pro-functional group, or isostere thereof, or may be metabolised to an active functional group. In some embodiments, at least R¹, R², R⁴, and R⁵ may be hydrophobic. For example, each of R¹, R², R⁴, and R⁵ may each, individually, be hydrogen, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. In some embodiments, R¹ and R³ may be a functional group capable of making a hydrogen bond with a functional group associated with the binding pocket of a RAS protein. For example, R¹ and R³ may each, individually, be hydrogen, hydroxyl, carboxyl, amino, alkyloxy, aryloxy, ketonyl, ester, or ether.

In some embodiments, R5 may be linear or branched C₂-C₁₀ alkyl. Thus, embodiments include compounds of general formula III:

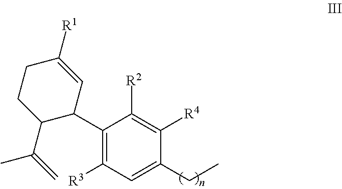

where R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres; R⁴ is hydrogen, substituted or unsubstituted alkyl, carboxyl, alkoxy, aryl, aryloxy, arylalkyl, halo or amino, or pharmaceutically acceptable isosteres; R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres, and n may an integer of 2 to 10 and the like. The compounds of Formula III encompass stereoisomers, salts, and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof.

In some embodiments, the compounds may be of general formula IV:

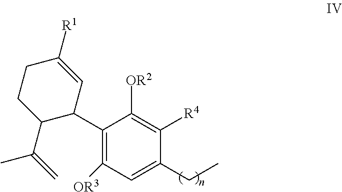

where R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; R² and R³ are each, individually, hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, or pharmaceutically acceptable isosteres; R⁴ is hydrogen, substituted or unsubstituted alkyl, carboxyl, alkoxy, aryl, aryloxy, arylalkyl, halo or amino, or pharmaceutically acceptable isosteres and n may an integer of 2 to 10 and the like. The compounds of Formula I encompass stereoisomers, salts, and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof.

In some embodiments, R⁶ or R⁷ and R³ as illustrated in Formula I may be covalently connected to produce a cyclized moiety. For example, embodiments include compounds of general Formula V:

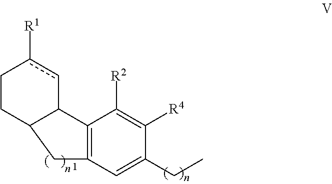

where R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; R² is hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres; R⁴ is hydrogen, substituted or unsubstituted alkyl, carboxyl, alkoxy, aryl, aryloxy, arylalkyl, halo or amino, or pharmaceutically acceptable isosteres, and n and n¹ are each, individually, an integer of 2 to 10 and the like. The compounds of Formula V encompass stereoisomers, salts, and solvates thereof. In some embodiments, R² may be a linear or branched, substituted or unsubstituted C₂-C₁₀ acyl having a carboxylic acid terminus thereby producing a dicarboxylic acid, and salts thereof.

In some embodiments, R⁶ or R⁷ and R³ as illustrated in Formula I may be covalently connected to produce a cyclized moiety. For example, embodiments include compounds of general Formula VI:

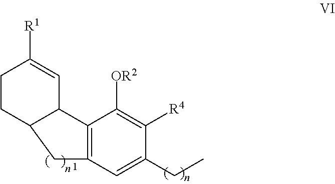

where R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² is hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, or pharmaceutically acceptable isosteres; R⁴ is hydrogen, substituted or unsubstituted alkyl, carboxyl, alkoxy, aryl, aryloxy, arylalkyl, halo, or amino or pharmaceutically acceptable isosteres. In various embodiments, n and n¹ are each, individually, an integer of 2 to 10 and the like. In some embodiments, the aliphatic chains created by n and n1 may include one or more heteroatoms such as oxygen, nitrogen, sulfur. Additionally, a heteroatom can occupy the position at which the heterocycle is attached to the remainder of the molecule. The compounds of Formula VI encompass stereoisomers, salts, and solvates thereof. In some embodiments, R² may be a linear or branched, substituted or unsubstituted C₂-C₁₀ alkyl having a carboxylic acid terminus thereby producing a dicarboxylic acid, and salts thereof.

In various embodiments, the compounds described above may be in a pharmaceutically acceptable salt form which, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and —N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted above are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

Further embodiments include compositions containing the compounds described above of Formulae I-VI or a pharmaceutically acceptable salt, ester, or salt of ester thereof and a pharmaceutically acceptable carrier, adjuvant, or vehicle. Such compositions may include an effective amount of the compound. An “effective amount” may inhibit RAS activity, slow cellular growth and proliferation, stop tumor growth, stop spread of cancerous cells and tissues from a site of malignancy. In various embodiments, the compositions of the invention may be formulated for administration to a patient in need of such composition.

Pharmaceutically acceptable carriers, adjuvants, or vehicles include not various compounds and compositions that allow for administration of the compounds without impacting the pharmacological activity of the compound with which it is formulated. Pharmaceutically acceptable carriers, adjuvants or vehicles that can be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In certain embodiments, the compositions may be administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution.

In some embodiments, fixed oils may be used as a solvent or suspending medium. Any bland fixed oil can be used for this purpose including synthetic mono-or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of the compound of various compounds described above may be combined with the carrier materials to produce a composition in a single dosage form will vary depending upon the host treated, the particular mode of administration. For example, in some embodiments, the compositions may be formulated to include the compounds in a dosage of between 0.01-100 mg/kg body weight, although a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. In some embodiments, the amount of an individual compound will depend on the pharmacological properties of the particular compound.

In some embodiments, the compositions described above may further include one or more pharmaceutically acceptable diluents, fillers, disintegrants, binders, lubricants, surfactants, emulsifiers, buffers, humectants, solubilizers, preservatives, colorants, plasticizers, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

For example in some embodiments, the composition may include a buffering agent. Buffering agents may be used to provide drug stability, to control the therapeutic activity of the drug substance, and to prevent the initial discomfort associated with injections. Suitable buffers include, but are not limited to, sodium bicarbonate, sodium citrate, citric acid, sodium phosphate, and the like and combinations thereof. When one or more buffers are utilized in the formulations of the invention, they may be combined with a pharmaceutically acceptable vehicle and present in an amount from about 0.1% (w/w) to about 20% (w/w).

In some embodiments, the composition may include an antioxidant. Such antioxidant may be, for example, butylated hydroxytoluene, ascorbic acid, ascorbyl palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

Broadly speaking, the formulations may be prepared by combining together the components of the formulation, as described herein, at a temperature and for a time sufficient to provide a pharmaceutically acceptable composition. For example, in some embodiments, the compositions components of the compositions may be dissolved, suspended, dispersed or otherwise mixed in a selected carrier or vehicle, at an effective concentration such that the condition to be treated is relieved or ameliorated.

Additional embodiments are directed to methods of treatment that include administering the compounds or compositions of the invention to a patient in need of treatment. In some embodiments, administering may occur after one or more symptoms have developed. In other embodiments, administering may be carried out in the absence of symptoms. For example, the patient in need of treatment may be an individual susceptible to a disease or malady, for example, an individual having a history or familial disposition to the disease or malady or that has been exposed to susceptibility factors, that can be treated using the compounds and compositions of the invention prior to the onset of symptoms. In some embodiments, administering may be carried out after symptoms have resolved, for example, to prevent or delay their recurrence.

In certain embodiments, the present invention provides a method of treating cancer or another proliferative disorder by administering a compound or composition of the invention to a patient with cancer or another proliferative disorder. In certain embodiments, the method of treating cancer or another proliferative disorder may include administering compounds and compositions of the present invention to a mammal, and in some embodiments, the mammal is a human. As used herein, the terms “inhibition of cancer” and “inhibition of cancer cell proliferation” refer to the inhibition of the growth, division, maturation or viability of cancer cells, and/or causing the death of cancer cells, individually or in aggregate with other cancer cells, by cytotoxicity, nutrient depletion, or the induction of apoptosis.

Examples of tissues containing cancerous cells whose proliferation is inhibited by the compounds and compositions described herein and against which the methods described herein are useful include but are not limited to breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gallbladder, rectum, salivary gland, adrenal gland, throat, esophagus, lymph nodes, sweat glands, sebaceous glands, muscle, heart, and stomach.

In some embodiments, the cancer treated by compounds or compositions of the invention include melanoma, liposarcoma, lung cancer, breast cancer, pancreatic cancer, prostate cancer, leukemia, kidney cancer, esophageal cancer, brain cancer, lymphoma or colon cancer. In certain embodiments, the cancer is a primary effusion lymphoma (PEL). In some embodiments, the cancer to be treated by compounds or compositions of the invention is one bearing an activated MAPK pathway. In some embodiments the cancer bearing an activated MAPK pathway is a melanoma. In some embodiments, the cancer treated by compounds or compositions of the invention is one associated with BRCA1 mutation. In some embodiments, the cancer treated by compounds or compositions of the invention may be a triple negative breast cancer or non-small cell lung cancer (NSCLC).

In some embodiments, the compounds and compositions, according to the method of the present invention, may be administered using any amount and any route of administration effective for treating or lessening the severity of a LKB1 or KRAS associated disease. In some embodiments, the LKB1 or KRAS associated disease is selected from hepatocellular carcinoma, LKB1 mutant cancers, LKB1 loss of heterozygosity (LOH) driven cancers, KRAS mutant cancers, Peutz-Jeghers syndrome (PJS), Cowden's disease (CD), and tuberous sclerosis (TS). In some embodiments, the LKB1 or KRAS associated disease may be a KRAS deficient lung cancer, colorectal cancer, melanoma, breast cancer, liposarcoma, and the like and combinations thereof. In such embodiments, the compounds and compositions, according to the method of the present invention, may be administered using any amount and any route of administration effective for treating or lessening the severity of a cancer, or inhibiting the growth of or inducing apoptosis in cancer cells.

In some embodiments, the compounds and compositions of the invention may be administered using any amount and any route of administration effective for treating or lessening the severity of a liver disease. In some embodiments, the liver disease is selected from hepatitis C, hepatocellular carcinoma, familial combined hyperlipidemia and non-alcoholic steatohepatitis (NASH), liver cancer, cholangiocarcinoma, angiosarcoma, hemangiosarcoma, progressive familial intrahepatic cholestasis, and the like and combinations thereof.

In certain embodiments, the compounds or composition of various embodiments described above may be administered in combination with another anti-cancer, cytotoxin, or chemotherapeutic agent, to a patient in need of treatment such as a patient having cancer or proliferative disorder. Anti-cancer or chemotherapeutic agents that can be administered in combination with the compounds and composition of the invention are note limited and include, but are not limited to, metformin, phenformin, buformin, imatinib, nilotinib, gefitinib, sunitinib, carfilzomib, salinosporamide A, retinoic acid, cisplatin, carboplatin, oxaliplatin, mechlorethamine, cyclophosphamide, chlorambucil, ifosfamide, azathioprine, mercaptopurine, doxifluridine, fluorouracil, gemcitabine, methotrexate, tioguanine, vincristine, vinblastine, vinorelbine, vindesine, podophyllotoxin, etoposide, teniposide, tafluposide, paclitaxel, docetaxel, irinotecan, topotecan, amsacrine, actinomycin, doxorubicin, daunorubicin, valrubicin, idarubicin, epirubicin, plicamycin, mitomycin, mitoxantrone, melphalan, busulfan, capecitabine, pemetrexed, epothilones, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Citrovorum Factor, Cladribine, Cortisone, Cosmegen®, CPT-11, Cytadren®, Cytosar-U®, Cytoxan®, Dacarbazine, Dacogen, Dactinomycin, Darbepoetin Alfa, Dasatinib, Daunomycin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab, ozogamicin, Gemzar Gleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-AR (Interferon Alfa-2a), Romiplostim, Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICER, Toposar®, Topotecan, Toremifene, Torisel®, Tositumomab, Trastuzumab, Treanda®, Tretinoin, Trexall™, Trisenox®, TSPA, TYKERBR, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xeloda®, Zanosar®, Zevalin™, Zinecard®, Zoladex®, Zoledronic acid, Zolinza, Zometa®, or combinations of any of the above. In some embodiments, a combination of 2 or more therapeutic agents may be administered together with compounds of the invention. In other embodiments, a combination of 3 or more therapeutic agents may be administered with compounds of the invention.

Other examples of agents the inhibitors of this invention may also be combined with include, without limitation: vitamins and nutritional supplements, cancer vaccines, treatments for neutropenia (e.g. G-CSF, filgrastim, lenograstim), treatments for thrombocytopenia (e.g. blood transfusion, erythropoietin), PI3 kinase (PI3K) inhibitors, MEK inhibitors, mTOR inhibitors, CPT1 inhibitors, AMPK activators, PCSK9 inhibitors, SREBP site 1 protease inhibitors, HMG CoA-reductase inhibitors, antiemetics (e.g. 5-HT3 receptor antagonists, dopamine antagonists, NK1 receptor antagonists, histamine receptor antagonists, cannabinoids, benzodiazepines, or anticholinergics), treatments for Alzheimer's Disease such as Aricept® and Exelon®; treatments for Parkinson's Disease such as L-DOPA/carbidopa, entacapone, ropinirole, pramipexole, bromocriptine, pergolide, trihexyphenidyl, and amantadine; agents for treating Multiple Sclerosis (MS) such as beta interferon (e.g., Avonex® and Rebif®), Copaxone®, and mitoxantrone; treatments for asthma such as albuterol and Singulair; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anticonvulsants, ion channel blockers, riluzole, and anti-Parkinsonian agents; agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs. In some embodiments, compounds of the present invention, or a pharmaceutically acceptable composition thereof, are administered in combination with antisense agents, a monoclonal or polyclonal antibody or an siRNA therapeutic.

The additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another. As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of formula I, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of about 0.01 mg/kg body weight to about 100 mg/kg body weight of the composition can be administered. In various embodiments, the inventive compounds or compositions containing such compounds may be administered at a therapeutically effective amount, for example, about 0.1 mg/kg, about 0.5 mg/kg, about 1.0 mg/kg, about 2.0 mg/kg, about 2.5 mg/kg, about 5 mg/kg, about 10 mg/kg, about 15 mg/kg, about 20 mg/kg, about 25 mg/kg, about 30 mg/kg, about 40 mg/kg, about 50 mg/kg, about 60 mg/kg, about 70 mg/kg, about 80 mg/kg, about 90 mg/kg, about 100 mg/kg or any dosage encompassed by these examples. In certain embodiments, the inventive compounds or compositions containing such compounds may be administered at a therapeutically effective amount, for example, about 0.5 mg/kg to about 90 mg/kg, about 1.0 mg/kg to about 70 mg/kg, about 2.0 mg/kg to about 50 mg/kg, about 2.5 mg/kg to about 25 mg/kg, about 5 mg/kg to about 20 mg/kg, or any range or individual dosage encompassed by these example ranges. In various embodiments, the amount of the compositions of the invention in compositions may be about 40 mg to about 800 mg, and in some embodiments, the amount of the compounds of the invention may be provided in an amount of about about 50 mg to about 600 mg, about 50 mg to about 500 mg, about 100 mg to about 400 mg, or any range or individual amount encompassed by these example ranges. Similarly, the amount of the chemotherapeutic agent in the compositions of the invention may be about 40 mg to about 800 mg, and in some embodiments, the amount of the chemotherapeutic may be provided in an amount of about about 50 mg to about 600 mg, about 50 mg to about 500 mg, about 100 mg to about 400 mg, or any range or individual amount encompassed by these example ranges

The amount of additional therapeutic agent present in the compositions of this invention will be no more than the amount that would normally be administered in a composition including that therapeutic agent as the only active agent. Thus, in those compositions which comprise an additional therapeutic agent, that additional therapeutic agent and the compound of this invention may act synergistically, and the amount of additional therapeutic agent in such compositions will be less than that required in a monotherapy utilizing only that therapeutic agent. In such embodiments, the amount of additional therapeutic agent in the presently disclosed compositions will range from about 50% to 100% of the amount normally present in a composition comprising that agent as the only therapeutically active agent.

The compositions of the invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

The exact amount of the compounds required will vary from subject to subject, and may depend, for example, on the species, age, and general condition of the subject, the severity of the disease, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly (orthoesters) and poly (anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type can be used as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may contain opacifying agents and can also be of a composition that releases the active ingredient(s) only in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that releases the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

In some embodiments, the compositions may be delivered topically by, for example, applying the compositions directly to skin of a patient in need for treatment at the location of the injury or disease. In other embodiments, the compositions may be applied locally to the skin of a patient before, for example, surgery, injections, or other medically necessary injury. In further embodiments, the compositions may be administered transdermally, percutaneously, or by microneedle injection. Administration can also be, for example, intravenous, intraperitoneal, subdermal, subcutaneous, intradermal, transcutaneous, intramuscular, oral, intra-joint, parenteral, intranasal, or by inhalation. Suitable sites of administration thus include, but are not limited to, the skin, bronchium, gastrointestinal tract, eye, buccal cavity, and ear.

In embodiments such as those described above, the compounds and compositions of the invention can be administered one or more times each day, and administering can be carried out for a period of at least 1 month, 2 months, 3 months, 4 months, 6 months, 8 months, 12 months, or indefinitely depending on the condition of the patient or disease or injury being treated. In some embodiments, the composition may be administered once, as needed, once daily, twice daily, three times a day, once a week, twice a week, every other week, every other day, or the like. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

### KRAS (G12c) Nucleotide Exchange Assay

To test the ability of the compounds encompassed by the invention to alter the GDP to GTP nucleotide exchange, four compounds (TV-1001, TV-1002, TV-1003, and TV-1004) were subjected to a cell free assay that assessed their ability to either increase or inhibit KRAS activity in a dose dependent fashion. The working hypothesis was that these compounds would increase KRAS activity.

KRAS nucleotide exchange assay was carried out using a KRAS (G12c) Nucleotide Exchange Assay Kit (BPS Bioscience, San Diego CA) with an Enspire Plate reader (Perkin Elmer, Waltham MA) according to manufacturer's protocol. KRAS (G12C) is one of the KRAS mutations that is found frequently in lung and colon cancers. The KRAS (G12C) Nucleotide Exchange Assay Kit uses BODIPY-GDP to monitor the GDP or GTP binding status. Increased fluorescence indicates that nucleotide exchange is occurring and KRAS (G12C) is activated. Lack of a fluorescent signal indicates that KRAS (G12C) remains inactive.

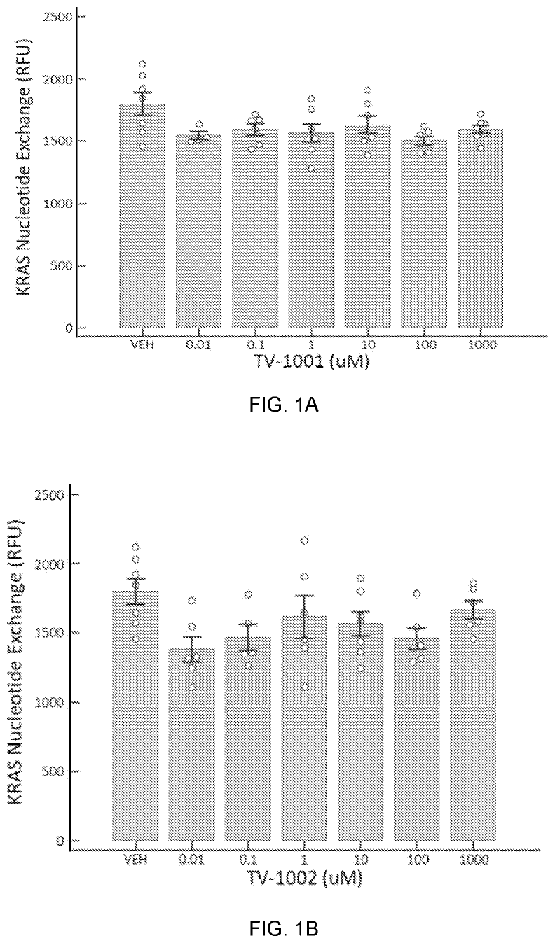

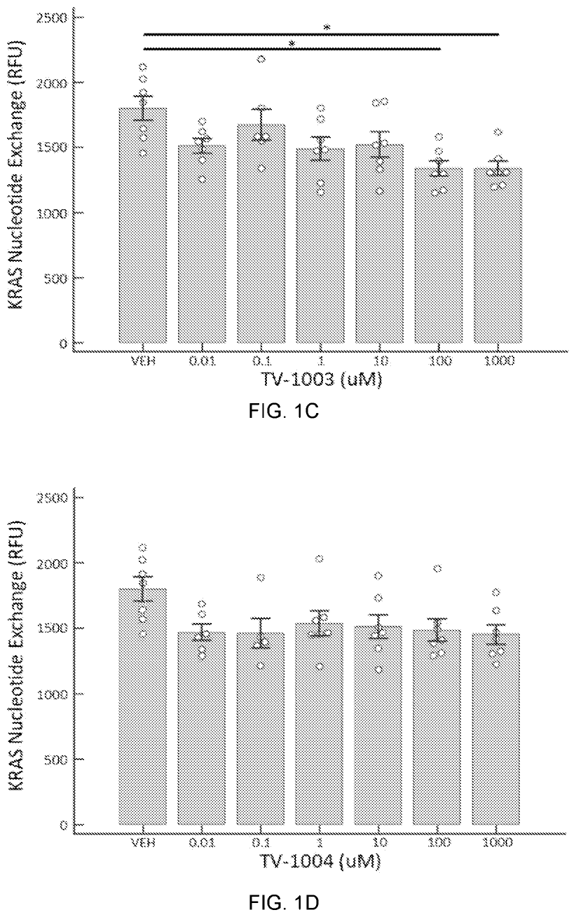

A total of 130 samples were tested including vehicle only and no drug controls. Each of the four compounds was diluted from 1:0.1 to 1:1000. The resulting rate of nucleotide exchange based on the change in fluorescence is shown in FIG. 1A-FIG. 1D.

These experiments indicate that all of the compounds tested do appear to have a direct effect on KRAS (G12C) with a dose dependent activation of nucleotide exchange. In particular, TV-1003 significantly increased KRAS (G12C) nucleotide exchange in a dose dependent manner. All other TV compounds trended towards an increase in KRAS activity without reaching a significant alteration from vehicle treatment. These data necessitate further experimentation in KRAS (G12C) mutant cell line models to evaluate their in vitro effect.

### Example 2

### IC50 Determination

To determine the ability of the compounds (TV-1001, TV-1002, TV-1003, TV-1004) to reduce the cell viability across a panel of 7 pancreatic cancer cell lines, a tenfold dilution scheme was used to generate an IC50 based on their dose dependent reduction of cell viability. Viability was measured using CellTiter-Glo 2.0 (Promega, Madison, WI), which measures cell viability by quantifying the concentration of ATP in each sample, an indicator of metabolically active cells.

Cells from each of the cell lines were aliquoted into a 96-well plate in a total of 50 μL per well. Cells were allowed to attach to the vessel for 48 hours before TV compounds were added to the plate. The compounds were diluted into appropriate growth media at a 2× the intended final concentration and 50 μL of the respective mixture was added to the respective treatment wells, bringing the total volume of each well to 100 μl. The vehicle group (VEH) for this study was 1% DMSO. After a 48 h incubation, 100 μl of CellTiter-Glo 2.0 (Promega, Madison, WI) reagent was added to each well. Plates were then incubated, with shaking for 2 min @RT. After 2 min 150 μl was taken from each well and transferred to a 96-well flat bottom opaque white plate that was incubated in the dark for 10 min. After 10 min plates were analyzed for luminescence by use of Enspire Plate reader (Perkin Elmer, Waltham MA).

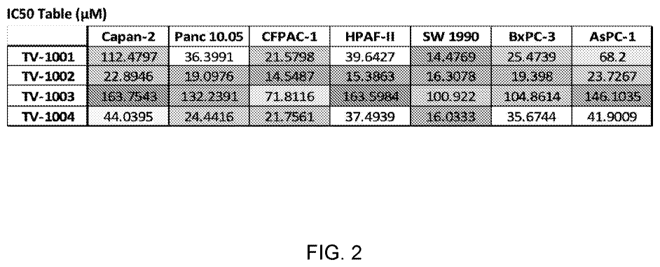

These experiments resulted in a successful identification of an IC50 for each compound with a dose dependent reduction in viability in each cell line with each compound. TV-1002 has shown to be the most potent with IC50s in the 15 μM to 25 μM range. See FIGS. 2. TV-1001 and TV-1003 were the least potent compounds with IC50s as high as 164 μM. Capan-2, AsPC-1, and Panc 10.05 were the most resistant to the TV compounds while CFPAC-1 and SW 1990 were the most sensitive.

These data indicate that the TV compounds are potent cytotoxic agents with applicability across pancreatic cancer as indicated by their similar effect across a panel of 7 cell lines with various mutational discrepancies. One cell line in this panel, BxPC-3, is a non-KRAS mutant and may also indicate the applicability of these compounds outside of the setting of mutant KRAS. Additionally, we observed what appeared to be growth arrest in cells treated with lower doses of TV-1003 which may imply a cell cycle arrest mechanism in addition to cytotoxicity.

### Example 3

### Hit Finder Assay

To determine the effect of combining of the test compounds with other chemotherapeutics, test compounds (TV-1001, TV-1002, TV-1003, and TV-1004) were administered with 7 FDA approved chemotherapeutics (gemcitabine, paclitaxel, cisplatin, oxaliplatin, 5-fluorouracil, capecitabine and irinotecan) using single doses of each agent in combination using the same procedure described in Example 2. All FDA approved compounds are at a concentration of 1 μM and all test compounds are at a concentration of 10 μM.

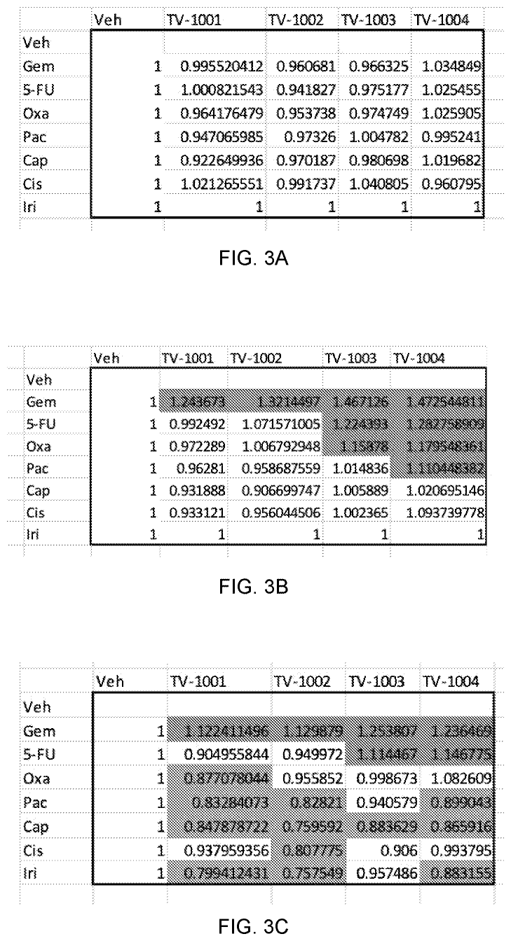

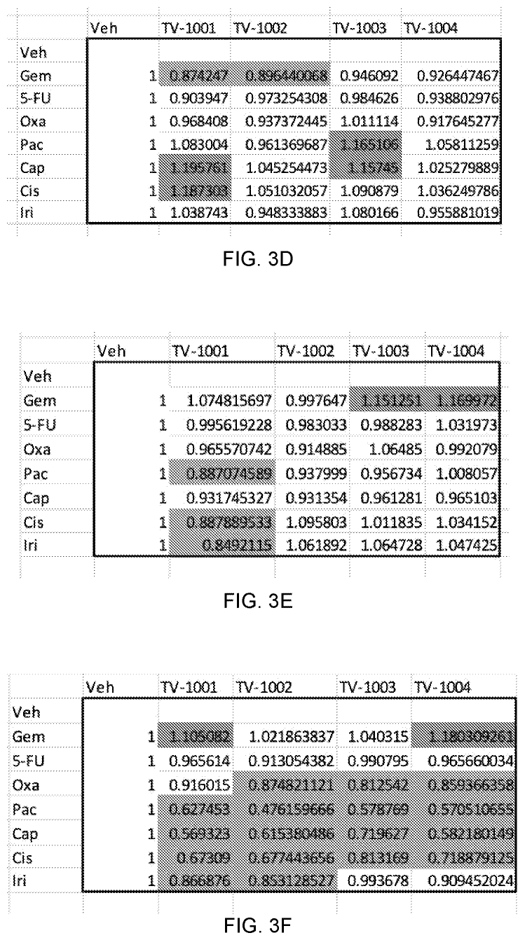

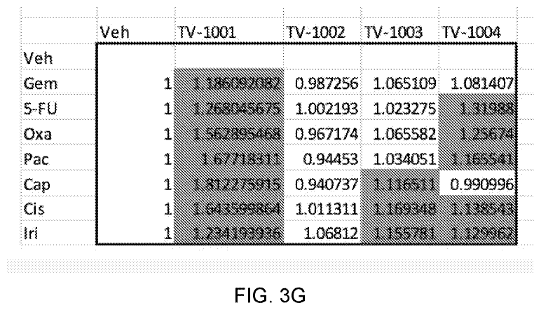

Drug-drug interactions that potentiate cell killing were analyzed in a two-step process. Cell viability was first calculated in relation to the vehicle control, arbitrarily setting the control as 100% viable. This calculation demonstrated the percent killing of each drug, alone or in combination. Once this was determined, the agent with the highest percentage of cell killing was set at 100%, with all combination treatments for that drug being a percentage of cell killing, in relation to the single drug. This calculation allows for the determination of drug combinations that increase or decrease the efficacy of cell killing by 10%. Red in FIG. 3A-FIG. 3G indicates a drug-drug interaction that resulted in less cell killing by at least 10%. Green indicates drug combinations that increased cell killing by at least 10%.

Drug-drug interactions that resulted in increased cell killing were observed in four of the cell lines (CFPAC-1, HPAF-II, SW 1990, and BxPC-3). The combinations were particularly effective for cell lines (CFPAC-1 and BxPC-3). In particular, test compounds TV-1002 and TV-1003 in combination with paclitaxel and oxaliplatin showed promise against cell lines CFPAC-1 and BxPC-3.

### Example 4

### Synergy Finder Assay

The objective of this study is to determine the combinatorial efficacy (antagonism, additivity, or synergy) of TV-1002 and TV-1003 with paclitaxel using multiple concentration ratios for the drug.

Chemotherapeutics were purchased from Cayman Chemical (Ann Arbor, MI). Dimethyl sulfoxide (DMSO) was purchased from Fisher Scientific (Waltham, MA). Phosphate Buffered Saline was purchased from Thermo Fisher Scientific (Waltham, MA). All compounds were diluted in the appropriate solvent, as indicated in the table below. Once dissolved, all compounds were subaliquotted to minimize effects from freeze-thaw cycles. All cell culture media and reagents were purchased and utilized as is indicated in Example 2. CellTiter-Glo 2.0 was purchased from Promega (Madison, WI)

Cell lines were seeded in a 96-well plate, according to the methods described in Example 2 and were allowed to attach to the vessel, prior to drug being added to the plate. Drug was diluted into appropriate growth media, prior to being added to the 96-well culture plate, bringing the total volume of each well to 100 μl. The vehicle group (VEH) for this study was 0.1% DMSO for all plates containing TV-1003 and 0.05% for all wells containing TV-1002. After drug treatment, cells were cultured as described in Example 2. After a 48 h incubation, 100 μl of CellTiter-Glo 2.0 reagent was added to each well. Plates were incubated with shaking for 2 min. After 2 min, 150 μl was taken from each well and transferred to a 96-well flat bottom opaque, white plate and incubated in the dark for 10 min. After 10 min plates were analyzed for luminescence by use of Enspire Plate reader (Perkin Elmer, Waltham MA). All synergy assays were carried out in CFPAC-1 (CRL-1918) and BxPC-3 (CRL-1687) cells in triplicate.

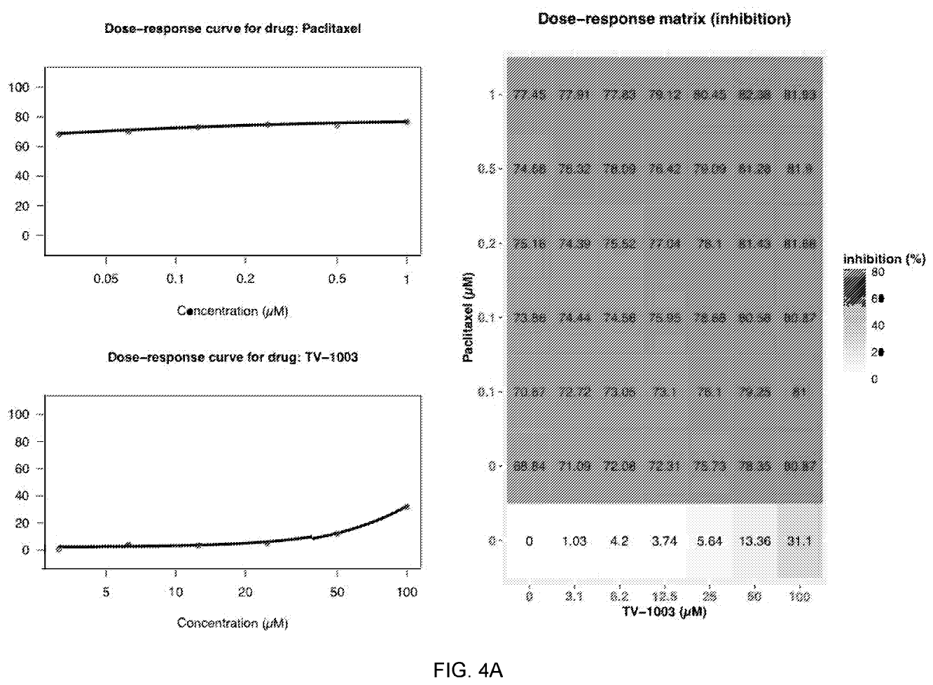

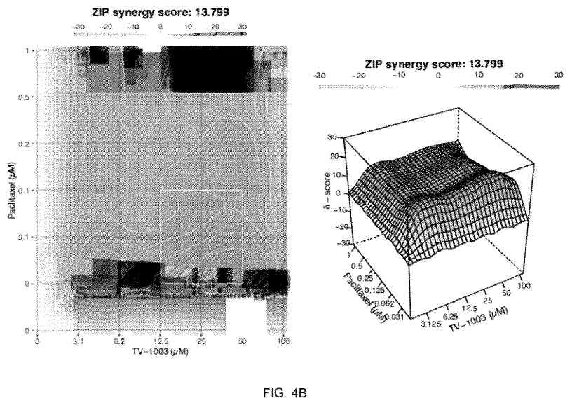

Cell viability was calculated as compared to the vehicle (VEH) control to determine the combinatorial effects of TV-1002 and TV-1003 with paclitaxel. It is worth noting that there was no significant change in viability with the vehicle control group, compared to untreated (BKG). Each cell line was analyzed for synergy after averaging three separate experiments. Paclitaxel was found to be synergistic with TV-1003 (synergy score >10) and additive for TV-1002 (synergy score >0, <10) in both cell lines. Representative synergy data is presented in FIG. 4, and synergy score calculations are presented in Table 1 (CFPAC-1 cell line) and Table 2 (BxPC-3 cell line).

TABLE 1
Synergy scores CFPAC-1

| | Synergy | Synergistic | |
|---|---|---|---|
| Combination | Score | Area Score | Method |
| TV-1002-Paclitaxel | 9.88 | 17.79 | Zip |
| TV-1003-Paclitaxel | 13.80 | 17.5 | Zip |

TABLE 2
Synergy scores BxPC-3

| | Synergy | Synergistic | |
|---|---|---|---|
| Combination | Score | Area Score | Method |
| TV-1002-Paclitaxel | 5.31 | 13.70 | Zip |
| TV-1003-Paclitaxel | 10.64 | 14.03 | Zip |

This study concluded that TV-1003, in combination with paclitaxel, synergistically killed CFPAC-1 and BxPC-3 cells. These promising data call for a more detailed analysis of these compounds for the treatment of KRAS mediated cancers, including mechanistic studies and in vivo efficacy studies.

1. A composition for treating cancers associated with RAS and KRAS mutations, comprising a therapeutically effective amount of a compound of general Formula I or stereoisomers, salts, and solvates thereof: 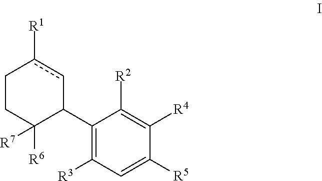
wherein:
R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres;
R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres;
R⁴ is hydrogen, hydroxyl, halogen, substituted or unsubstituted alkyl, carboxyl, alkoxy, aryl, aryloxy, arylalkyl, or pharmaceutically acceptable isosteres;
R⁵ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; and
R⁶ and R⁷ are each, individually, hydrogen, hydroxyl, halogen, substituted or unsubstituted alkyl, substituted or unsubstituted alkenyl, substituted or unsubstituted alkynyl, carboxyl, alkoxy, aryl, aryloxy, arylalkyl, amino, or pharmaceutically acceptable isosteres;
wherein cycloalkyl moieties can include optional double bonds indicated by dashed lines;
a therapeutically effective amount of a chemotherapeutic agent; and
a pharmaceutically acceptable excipient.

2. The composition of claim 1, wherein the chemotherapeutic agent is selected from the group consisting of metformin, phenformin, buformin, imatinib, nilotinib, gefitinib, sunitinib, carfilzomib, salinosporamide A, retinoic acid, cisplatin, carboplatin, oxaliplatin, mechlorethamine, cyclophosphamide, chlorambucil, ifosfamide, azathioprine, mercaptopurine, doxifluridine, fluorouracil, gemcitabine, methotrexate, tioguanine, vincristine, vinblastine, vinorelbine, vindesine, podophyllotoxin, etoposide, teniposide, tafluposide, paclitaxel, docetaxel, irinotecan, topotecan, amsacrine, actinomycin, doxorubicin, daunorubicin, valrubicin, idarubicin, epirubicin, plicamycin, mitomycin, mitoxantrone, melphalan, busulfan, capecitabine, pemetrexed, epothilones, 13-cis-retinoic acid, 2-CdA, 2-chlorodeoxyadenosine, 5-azacitidine, 5-fluorouracil, 5-FU, 6-mercaptopurine, 6-MP, 6-TG, 6-thioguanine, abraxane, accutane®, actinomycin-D, adriamycin®, adrucil®, afinitor®, agrylin®, ala-cort®, aldesleukin, alemtuzumab, ALIMTA, alitretinoin, alkaban-AQ®, alkeran®, all-transretinoic acid, alpha interferon, altretamine, amethopterin, amifostine, aminoglutethimide, anagrelide, anandron®, anastrozole, arabinosylcytosine, ara-C, aranesp®, aredia®, arimidex®, aromasin®, arranon®, arsenic trioxide, arzerra™, asparaginase, ATRA, avastin®, azacitidine, BCG, BCNU, bendamustine, bevacizumab, bexarotene, BEXXAR®, bicalutamide, BiCNU, blenoxane®, bleomycin, bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Citrovorum Factor, Cladribine, Cortisone, Cosmegen®, CPT-11, Cytadren®, Cytosar-U®, Cytoxan®, Dacarbazine, Dacogen, Dactinomycin, Darbepoetin Alfa, Dasatinib, Daunomycin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab, ozogamicin, Gemzar Gleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xeloda®, Zanosar®, Zevalin™, Zinecard®, Zoladex®, Zoledronic acid, Zolinza, Zometa®, and combinations thereof.

3. The composition of claim 1, wherein the chemotherapeutic agent is selected from the group consisting of gemcitabine, paclitaxel, cisplatin, oxaliplatin, 5-fluorouracil, capecitabine, and irinotecan.

4. The composition of claim 1, wherein the therapeutically effective amount of the compositions is about 0.01 to about 100 mg/kg body weight.

5. The composition of claim 1, wherein the composition comprises about 40 mg to about 800 mg of a compound of Formula 1.

6. The composition of claim 1, wherein the composition comprises about 40 mg to about 800 mg of a chemotherapeutic agent.

7. A method for treating cancer comprising, administering to a patient in need of treatment a therapeutically effective amount of a compound of general Formula I or stereoisomers, salts, and solvates thereof:
cyclophosphamide, chlorambucil, ifosfamide, azathioprine, mercaptopurine, doxifluridine, fluorouracil, gemcitabine, methotrexate, tioguanine, vincristine, vinblastine, vinorelbine, vindesine, podophyllotoxin, etoposide, teniposide, tafluposide, paclitaxel, docetaxel, irinotecan, topotecan, amsacrine, actinomycin, doxorubicin, daunorubicin, valrubicin, idarubicin, epirubicin, plicamycin, mitomycin, mitoxantrone, melphalan, busulfan, capecitabine, pemetrexed, epothilones, 13-cis-retinoic acid, 2-CdA, 2-chlorodeoxyadenosine, 5-azacitidine, 5-fluorouracil, 5-FU, 6-mercaptopurine, 6-MP, 6-TG, 6-thioguanine, abraxane, accutane®, actinomycin-D, adriamycin®, adrucil®, afinitor®, agrylin®, ala-cort®, aldesleukin, alemtuzumab, ALIMTA, alitretinoin, alkaban-AQ®, alkeran®, all-transretinoic acid, alpha interferon, altretamine, amethopterin, amifostine, aminoglutethimide, anagrelide, anandron®, anastrozole, arabinosylcytosine, ara-C, aranesp®, aredia®, arimidex®, aromasin®, arranon®, arsenic trioxide, arzerra™, asparaginase, ATRA, avastin®, azacitidine, BCG, BCNU, bendamustine, bevacizumab, bexarotene, BEXXAR®, bicalutamide, BiCNU, blenoxane®, bleomycin, bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Citrovorum Factor, Cladribine, Cortisone, Cosmegen®, CPT-11,Cytadren®, Cytosar-U®, Cytoxan®, Dacarbazine, Dacogen, Dactinomycin, Darbepoetin Alfa, Dasatinib, Daunomycin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab, ozogamicin, Gemzar 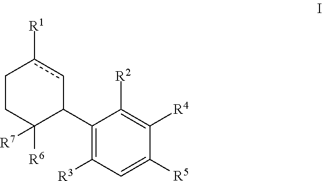
wherein:
R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres;
R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres;
R⁴ is hydrogen, hydroxyl, halogen, substituted or unsubstituted alkyl, carboxyl, alkoxy, aryl, aryloxy, arylalkyl, or pharmaceutically acceptable isosteres;
R⁵ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; and
R⁶ and R⁷ are each, individually, hydrogen, hydroxyl, halogen, substituted or unsubstituted alkyl, substituted or unsubstituted alkenyl, substituted or unsubstituted alkynyl, carboxyl, alkoxy, aryl, aryloxy, arylalkyl, amino, or pharmaceutically acceptable isosteres;
wherein cycloalkyl moieties can include optional double bonds indicated by dashed lines.

8. The method of claim 7, wherein the composition further comprises a therapeutically effective amount of a chemotherapeutic agent selected from the group consisting of metformin, phenformin, buformin, imatinib, nilotinib, gefitinib, sunitinib, carfilzomib, salinosporamide A, retinoic acid, cisplatin, carboplatin, oxaliplatin, mechlorethamine, Gleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xeloda®, Zanosar®, Zevalin™, Zinecard®, Zoladex®, Zoledronic acid, Zolinza, Zometa®, and combinations thereof.

9. The method of claim 8, wherein a therapeutically effective amount of compound of general Formula I or stereoisomers, salts, and solvates thereof and a chemotherapeutic agent is 0.01 mg/kg to about 50 mg/kg of subject body weight per day.

10. The method of claim 8, wherein the chemotherapeutic agent is selected from the group consisting of gemcitabine, paclitaxel, cisplatin, oxaliplatin, 5-fluorouracil, capecitabine, and irinotecan.

11. The method of claim 7, further comprising repeating the step of administering the compound.

12. The method of claim 7, wherein the cancer being treated is selected from the group consisting of melanoma, liposarcoma, lung cancer, breast cancer, prostate cancer, pancreatic cancer, leukemia, kidney cancer, esophageal cancer, brain cancer, lymphoma, colon cancer, and combinations thereof.

13. The method of claim 7, wherein the cancer being treated is pancreatic cancer.

14. The method of claim 7, wherein the cancer is a KRAS associated disease.

15. The method of claim 12, wherein the KRAS associated disease is selected from the group consisting of hepatocellular carcinoma, LKB1 mutant cancers, LKB1 loss of heterozygosity (LOH) driven cancers, KRAS mutant cancers, Peutz-Jeghers syndrome (PJS), Cowden's disease (CD), tuberous sclerosis (TS), and combinations thereof.

16. A composition comprising a therapeutically effective amount of:
a chemotherapeutic agent selected from the group consisting of gemcitabine, paclitaxel, cisplatin, oxaliplatin, 5-fluorouracil, capecitabine, and irinotecan; and
a therapeutically effective amount of a compound of general Formula I or stereoisomers, salts, and solvates thereof: 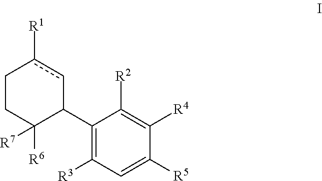
wherein:
R¹ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres;
R² and R³ are each, individually, hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, or pharmaceutically acceptable isosteres;
R⁴ is hydrogen, hydroxyl, halogen, substituted or unsubstituted alkyl, carboxyl, alkoxy, aryl, aryloxy, arylalkyl, or pharmaceutically acceptable isosteres;
R⁵ is hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, or pharmaceutically acceptable isosteres; and
R⁶ and R⁷ are each, individually, hydrogen, hydroxyl, halogen, substituted or unsubstituted alkyl, substituted or unsubstituted alkenyl, substituted or unsubstituted alkynyl, carboxyl, alkoxy, aryl, aryloxy, arylalkyl, amino, or pharmaceutically acceptable isosteres;
wherein cycloalkyl moieties can include optional double bonds indicated by dashed lines.

17. The composition of claim 16, wherein the therapeutically effective amount of the compositions is about 0.01 to about 100 mg/kg body weight.

18. The composition of claim 16, wherein the composition comprises about 40 mg to about 800 mg of a compound of Formula 1.

19. The composition of claim 16, wherein the composition comprises about 40 mg to about 800 mg of a chemotherapeutic agent.
